(19) United States
(12) Patent Application Publication
(10) Pub. No.: US 2007/0219534 A1
(43) Pub. Date: Sep. 20, 2007

(54) Modular chest drainage design and assembly method

(76) Inventors: Trinh D. Phung, Attleboro, MA (US); Erich A. Dreyer, Bellingham, MA (US); Douglas W. Moore, Westwood, MA (US)

Correspondence Address: BAKER & HOSTETLER LLP, WASHINGTON SQUARE, SUITE 1100, 1050 CONNECTICUT AVE. N.W., WASHINGTON, DC, 20036-5304, US

(21) Appl. No.: 11/377,551

(22) Filed: Mar. 17, 2006

(51) Int. Cl. A61M 27/00

(52) U.S. Cl. 604/541

(57) ABSTRACT

A modular chest drainage assembly is provided, having a collection module formed from a first base mold. The collection module defines a collection chamber having a patient fluid intake port. The assembly further includes a flow control module formed from a second base mold. A separate flow coupling is attachable to the collection module and flow control module to place the two modules in fluid communication. A pre-assembled pressure regulation module is coupled to the flow control module to regulate the pressure inside the collection module and flow control module. The assembly further includes a face plate, to which the collection module and flow control module are bonded such that the flow control module defines a first fluid flow pathway from the flow coupling to a suction port on the flow control module, for transmitting a suction flow from the collection chamber to the suction port.

### FIELD OF THE INVENTION

The present invention relates generally to medical devices. More particularly, the present invention relates to a chest drainage apparatus and method of assembly thereof.

### BACKGROUND OF THE INVENTION

Chest drainage devices and systems and more particularly suction drainage systems and devices for removing gases and/or liquids from medical patients, such as from the pleural cavity, by means of a pressure differential, are well known in the art. For many years, the standard apparatus for performing the evacuation of the pleural cavity was a drainage system known as the “3-bottle set-up” which includes a collection bottle, a water seal bottle, and a suction control bottle. A catheter runs from the patient's pleural cavity to the collection bottle, and the suction bottle is connected by a tube to a suction source. The three bottles are connected in series by various tubes to apply suction to the pleural cavity to withdraw fluid and air and thereafter discharge the same into the collection bottle. Gases entering the collection bottle bubble through water in the water seal bottle. The water in the water seal also usually prevents the back flow of air into the chest cavity. Suction or “negative” pressure is usually provided by a central vacuum supply in a hospital so as to permit withdrawal of fluids such as blood, water and gas from a patient's pleural cavity by establishing a pressure differential between the suction source and the internal pressure in the patient.

The 3-bottle set-up lost favor with the introduction of an underwater seal drainage system first sold under the name “Pleur-evac”® in 1966 by Deknatel Inc. U.S. Pat. Nos. 3,363,626; 3,363,627; 3,559,647; 3,683,913; 3,782,497; 4,258,824; and U.S. Pat. No. Re. 29,877 are directed to various aspects of the Pleur-evac® system, which over the years has provided improvements that eliminated various shortcomings of the 3-bottle set-up. These improvements have included the elimination of variations in the 3-bottle set-up that existed between different manufacturers, hospitals and hospital laboratories. A principal feature of the Pleur-evac® system is the use of a single, unitary, preformed, self-contained unit that embodies the 3-bottle techniques. The desired values of suction are generally established by the levels of water in a suction control chamber. These levels are filled according to specified values prior to the application of the system to the patient. A special valve referred to as the “High Negativity Pressure Relief Valve” (HNPRV) is included which is employed when the patient's ‘negativity’ becomes sufficient to threaten loss of the water seal. Also, a “Positive Pressure Release Valve” (PPRV) in the large arm of the water seal chamber works to prevent a tension pneumothorax when pressure in the large arm of the water seal exceeds a prescribed value because of suction malfunction, accidental clamping or occlusion of the suction tube. The Pleur-evac® system is disposable and helps in the battle to control cross-contamination.

One problem with the existing Pleur-evac® system and other known chest drainage devices and assemblies is that, due to the large number of constituent functioning elements in the device, such as the water seal, suction control, pressure or indicator manometer, and the like, when any one part of the overall assembly fails, the entire device is rendered useless. That is, because of the unitary construction and assembly of the chest drainage devices, replacement, repair, or redesign of any one component of the assembly is difficult. In addition, the unitary structure of the known chest drainage devices leads to increased construction and manufacturing costs, as large molds need to be made to produce the devices, which can be very expensive to replace or alter should the design of the apparatus change.

As listed above, various types of drainage apparatuses have been developed for use in draining fluids from the pleural cavity of a human being in a clean and aseptic environment. Examples of these drainage apparatuses are disclosed further in U.S. Pat. Nos. 3,757,783, 3,783,870, 3,853,128, 3,924,624, 3,946,735, 4,195,633, and 4,289,158. In U.S. Pat. No. 3,924,624 to Schachet, there is shown a modular device for chest drainage. However, this device has each of the chambers, which are separate bodies, connected solely to each other and requires a specific type of structural connection between the chambers. The structure of the aforesaid U.S. Pat. No. 3,924,624 to Schachet relies upon plastic tubing to provide the air communicating connections between the chambers. The plastic tubing has the possibility of ceasing to maintain the desired seal between the chambers so that the aseptic environment may be lost.

U.S. Pat. No. 4,465,483 to Weilbacher provides for another modular apparatus for chest drainage. However the Weilbacher patent provides a common support means for each of the separate plastic bodies making up an apparatus similar to the traditional 3-bottle set-up, with each body being disposed at a specific location on the support means. Thus there is a common connecting means between the various bodies rather than having the bodies connected to each other directly for their sole connections. All of the separate bodies are supported from the common or single support means, which has means providing air communication between the various chambers.

However the prior art discussed above still has several drawbacks and presents numerous disadvantages. First, the known modular assemblies are overly complex and involve unnecessary parts and elements which can lead to failure or malfunctioning of the device. The common support element in the Weilbacher patent is a prime example of such a drawback. Further, the method of assembly and manufacture of the various elements of the known chest drainage devices is too expensive and time-consuming. For the well-known chest drainage devices, the unit is traditionally assembled with all internal components placed into a single molded “back” or base component, made usually from a single prefabricated “base” mold. Such back or base molds can be large and very expensive. Changes or repairs to the mold are difficult because of production requirements. Typically the base mold would have to be utilized continuously during the production phase of the manufacturing cycle. When a design change or repair is needed all production is stopped. Repairs and modifications are very difficult due to the size and complexity of the mold.

It is desirable therefore, to provide a modular apparatus which would provide a more robust and cost-effective way of assembling and producing a chest drainage device. It is also desirable to provide a modular apparatus for chest drainage which improves upon the afore-discussed deficiencies of the prior art.

### SUMMARY OF THE INVENTION

The foregoing needs are met, to a great extent, by the present invention, wherein in one aspect an apparatus is provided that in some embodiments provides a modular apparatus which would provide a more robust and cost-effective way of assembling and producing a chest drainage device.

In accordance with one embodiment of the present invention, a modular chest drainage assembly is provided, having a collection module formed from a first base mold. The collection module defines a collection chamber having a patient fluid intake port. The assembly further includes a flow control module formed from a second base mold. A separate flow coupling is attachable to the collection module and flow control module to place the two modules in fluid communication. A pre-assembled pressure regulation module is coupled to the flow control module to regulate the pressure inside the collection module and flow control module. The assembly further includes a face plate, to which the collection module and flow control module are bonded such that the flow control module defines a first fluid flow pathway from the flow coupling to a suction port on the flow control module, for transmitting a suction flow from the collection chamber to the suction port.

In accordance with another aspect of the present invention, a modular chest drainage device is provided, having a collection module defining a fluid collection chamber having an exit port for transmitting a suction flow out of the collection chamber. A flow control module defines an entry port for receiving the suction flow from the collection chamber, a suction port for coupling to a suction source, and a pressure regulation flow intake port. A flow coupling is attached between the exit port and the entry port. A pressure regulation module is sealingly coupled to the pressure regulation flow intake port, and has an adjustable valve assembly for regulating a pressure regulation flow into the flow control module from an ambient air intake port. The collection module and flow control module are attached to a face plate to form: (i) a first fluid flow pathway from the entry port to the suction port for transmitting the suction flow from the collection chamber to the suction port, and (ii) a second fluid flow pathway from the pressure regulation flow intake port to the suction port for transmitting the pressure regulation flow, wherein the mixing of the suction flow and pressure regulation flow provides for regulation of pressure inside the collection chamber.

In accordance with yet another aspect of the present invention, a method of assembling a chest drainage device is provided. A collection module is formed from a first base mold, the collection module defining a collection chamber having a patient fluid intake port. A flow control module is formed from a second base mold. An adjustable pressure regulation module is assembled. A face plate is formed. The collection module and flow control module are bonded to the face plate such that the flow control module defines a first fluid flow pathway from a flow entry port to a suction port on the flow control module, for transmitting a suction flow from the collection chamber to the suction port. The adjustable pressure regulation module is coupled to the flow control module to allow regulation of the pressure inside the collection module and flow control module.

There has thus been outlined, rather broadly, certain embodiments of the invention in order that the detailed description thereof herein may be better understood, and in order that the present contribution to the art may be better appreciated. There are, of course, additional embodiments of the invention that will be described below and which will form the subject matter of the claims appended hereto.

In this respect, before explaining at least one embodiment of the invention in detail, it is to be understood that the invention is not limited in its application to the details of construction and to the arrangements of the components set forth in the following description or illustrated in the drawings. The invention is capable of embodiments in addition to those described and of being practiced and carried out in various ways. Also, it is to be understood that the phraseology and terminology employed herein, as well as the abstract, are for the purpose of description and should not be regarded as limiting.

As such, those skilled in the art will appreciate that the conception upon which this disclosure is based may readily be utilized as a basis for the designing of other structures, methods and systems for carrying out the several purposes of the present invention. It is important, therefore, that the claims be regarded as including such equivalent constructions insofar as they do not depart from the spirit and scope of the present invention.

### BRIEF DESCRIPTION OF THE DRAWINGS

FIG. 1 is a perspective view illustrating the separate components of a modular chest drainage device prior to final assembly, according to one embodiment of the invention.

FIG. 2 is a top view of the assembly shown in FIG. 1.

FIG. 3 is a schematic front view of a modular chest drainage device similar to that shown in FIG. 1, shown as assembled without the face plate.

FIG. 4 is a perspective view illustrating a flow control module of the present invention in accordance with one embodiment.

FIG. 5 is a perspective view illustrating the separate components of a modular chest drainage device prior to final assembly, according to another embodiment of the invention.

### DETAILED DESCRIPTION

The invention will now be described with reference to the drawing figures, in which like reference numerals refer to like parts throughout. An embodiment in accordance with the present invention provides a modular chest drainage device including several components or modules which can be separately designed, made, and then assembled together. The modules include a collection module having a collection chamber which includes a flow intake port for receiving body fluids from a patient, such as blood or air from a pleural cavity during chest surgery. Further included is a “flow control” module having one or more flow passageways, valves, water seals, manometers, check valves, or other flow control elements which can be used to control the flow, pressure, and other conditions in the overall apparatus, including the collection chamber of the collection module. The flow control module has a suction port that can be coupled to a source of suction which provides the source means for drawing fluids with the device from a body. A pressure regulation module having an adjustable valve assembly therein can be attached to the flow control module to regulate the pressure inside of the apparatus, as further described herein. The various modules are assembled together with a face plate having various markers, labels, and gradations which present the front operating interface for the unit. When the flow control module and collection module are bonded to the face plate, distinct flow passageways are formed which can be used to regulate the suction, drainage, and fluid collection functions of the apparatus. By using modular components which can be easily replaced or redesigned, the overall assembly can be much more cheaply and efficiently manufactured and utilized as compared to other known chest drainage devices.

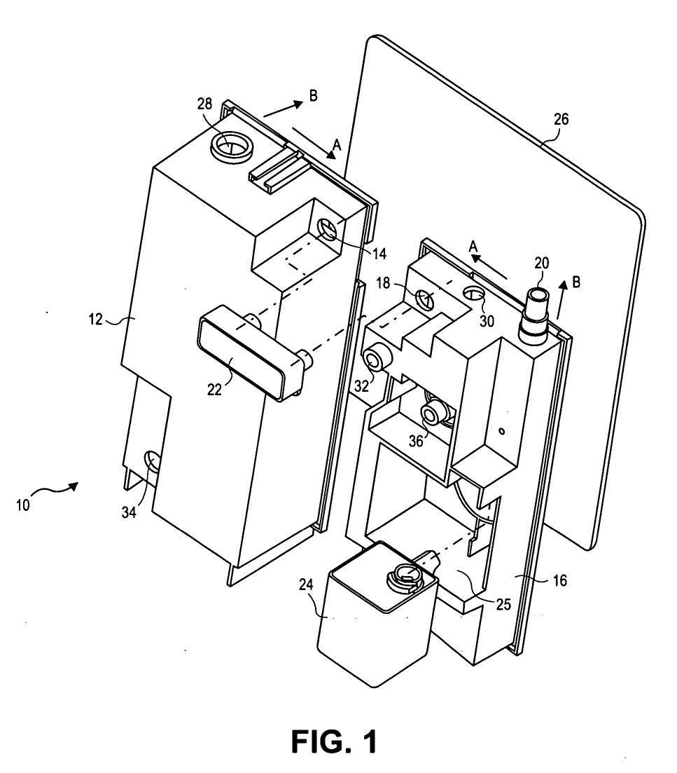

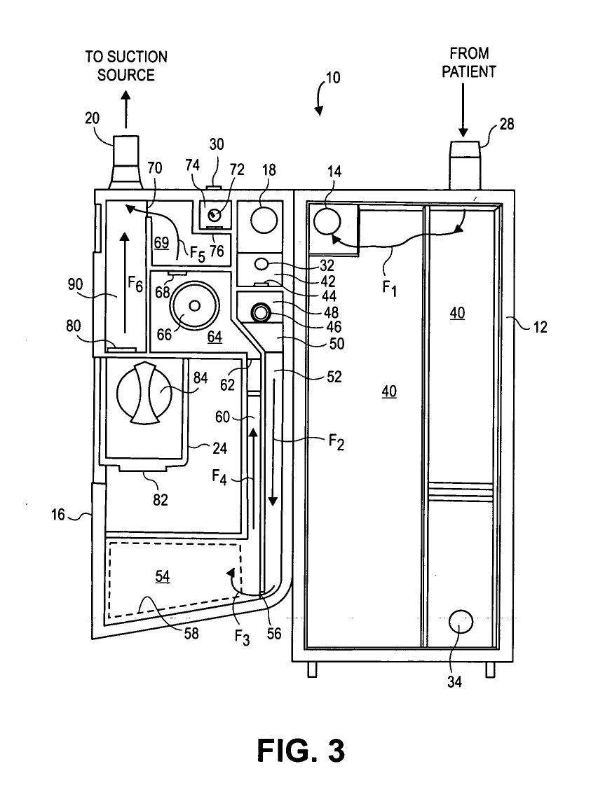

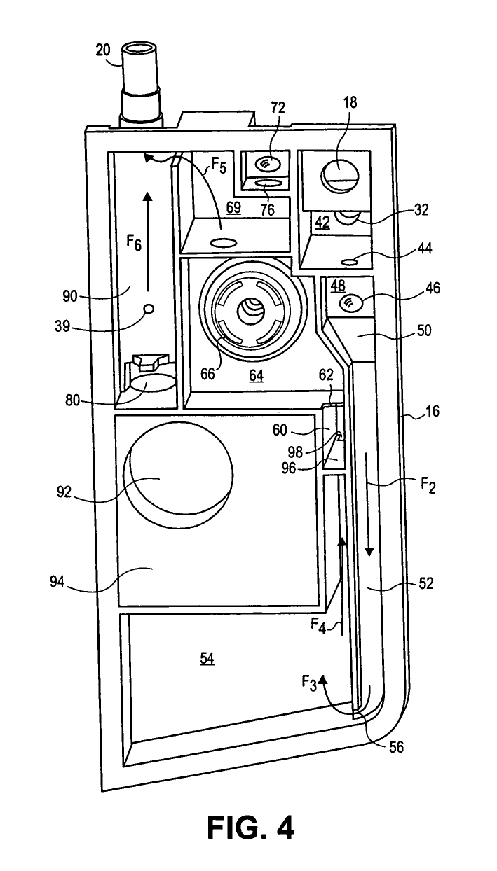

An embodiment of the present inventive apparatus is illustrated in FIG. 1. A modular chest drainage device 10 includes a collection module 12 defining a fluid collection chamber inside of it and having an exit port 14 for transmitting a suction flow out of the collection chamber. A ‘flow control’ module 16 defines an entry port 18 for receiving the suction flow from the collection chamber, a suction port 20 for coupling to a suction source (not shown), and a pressure regulation flow intake port (shown in FIGS. 3 and 4). A flow coupling 22 is provided between the exit port 14 and the entry port 18. A pressure regulation module 24 is sealingly coupled to the pressure regulation flow intake port on the flow control module 16 and can be positioned in an enclosure 25 defined by the walls and geometry of the flow control module 16 as shown in FIG. 1. The pressure regulation module 24 has an adjustable valve assembly therein for regulating a pressure regulation flow into the flow control module 16 from an ambient air intake port provided on the pressure regulation module 24. A face plate 26 is provided, wherein the collection module 12 and flow control module 16 are first aligned next to each other as in arrows A and then attached to face plate 26 as in arrows B so that the assembly can form multiple flow pathways, as will be illustrated in further detail below.

The collection module includes a fluid intake port 28 for receiving fluids from a patient. A catheter, tube, or similar device can be coupled to the fluid intake port 28 in a variety of ways as is well known in the art. An ambient airport 30 is included on the flow control module 16 as part of a positive pressure relief valve element therein. A filling valve 32, such as a grommet or needle-less fill valve with a lure type fitting, is provided on the flow control module 16 for injecting fluids into the module for filling a manometer chamber or water seal chamber that is needed to control the backflow of gases and to indicate pressure, flow, or breathing, as further explained below. A re-infusion port 34 is provided on the collection module 12 for allowing collected body fluids to be returned to a patient by a re-infusion line. A high negativity pressure relief valve 36 is also provided on the flow control module 16 to prevent excessive negative pressures from building in the device.

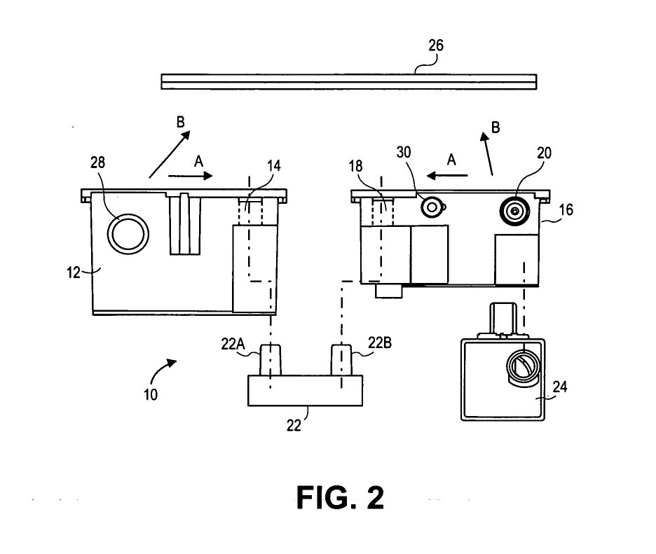

FIG. 2 is a top view of the assembly shown in FIG. 1. After the collection module 12 and flow control module 16 are aligned in the direction of arrows A to be positioned right next to one another, the flow coupling 22 is attached or coupled, permanently or detachably, to the collection module 12 through the flow exit port 14 with tubular extension 22a, and to the flow control module 16 through flow entry port 18 with tubular extension 22b. Thus, fluid or pressure communication, or a flow pathway, is established between the collection chamber inside of the collection module 12 and the flow pathways inside of flow control module 16.

FIG. 3 is a schematic front view of a modular chest drainage device 10 similar to that shown in FIG. 1, shown as assembled without the face plate. Fluid entering the device 10 from a patient first passes through the fluid intake port 28 and enters the collection chamber 40 defined inside the walls of the collection module 12. The collection chamber 40 can be made up of any number of compartments or sub-compartments, as is well known in the art, and can vary in size depending on the nature of the patient body to which the chest drainage device is attached: i.e. adult vs. pediatric sizes. Suction pressures established throughout the device 10 are also present in the collection chamber 40 such that gases entering the collection chamber 40 are passed out of the chamber through an exit port 14, while the liquid matter in the fluids captured inside of the collection chamber 40 remains trapped inside the chamber. Suction pressure is thereby ‘transmitted’ throughout the collection chamber via port 14, such that a ‘suction flow’ F1 is established between the intake port 28 and exit port 14. As used herein, the term ‘suction flow’ shall mean either a flow of gases or fluids from one point to another driven by a source of suction, or a flow in the direction of a negative pressure gradient, or an actual negative pressure gradient itself.

A manually triggered high pressure negativity relief valve can be fitted in the body of the collection module 12 to place the chamber 40 in communication with external ambient room air when excessive negative pressure builds in the device or patient. Said valve can have any of a variety of known manual triggering mechanisms and user interfaces.

After exiting the collection chamber 40, the suction flow is transmitted though the flow coupling 22 and enters the flow control module 16 through entry port 18. The flow then proceeds downwards according to the orientation of view in FIG. 3, into sub-compartment 42 which is in communication with the fill valve 32. The flow then passes through a hole 44 having a valve-seat shaped on its underside, under which a ball element 46 is disposed in another sub-compartment 48. The flow passes though this sub-compartment 48 past a ramped funnel compartment 50 into an arm 52 which, when filled with fluid, serves as part of a water seal element, which can be filled with fluid injected from fill port 32. The flow then proceeds in the direction F2 though the water seal element, which can also function as a breathing indicator manometer, such when the pleural pressure inside the patient as transmitted through the collection chamber 40 is highly negative, the water level in arm 52 rises opposite the direction F2 and causes the ball element 46 to drift upwards towards valve seat 44. The ball 46 and valve seat 44 are therefore a type of check valve. When the pleural pressure is more positive, the water level in the arm 52 drops. The breathing of a patient can also be monitored by the reciprocating up and down movement of the water level in arm 52 such that the water seal/manometer element functions as a breathing indicator.

Thus, the ‘suction flow’ can be transmitted along arrow F2 through the manometer in arm 52 into the water seal chamber 54 via flow arrow F3 which enters through a narrow opening 56 at the bottom of arm 52. An air leak indicator and metering element 58 can be included in chamber 54 as is well known in the art. Flow can then continue along pathway F4 through another passage or arm 60, past another opening 62, and into chamber 64. A high negativity pressure relief valve 66 is disposed on the flow control module 16 to place chamber 64 in fluid communication with ambient air outside the device when the pressure inside said chamber 64 exceeds a pre-determined negative pressure (gauge or absolute, as the case may be). The flow proceeds though another opening 68 into chamber 69 and along arrow F5 past an opening 70 and into the suction port 20 for capture by the suction source. Thus, the ‘suction flow’ or suction pressure can be transmitted through the device 10, from intake port 28 to exit 20. Opening 68 can also be fitted with a one-way check valve permitting suction flow from chamber 64 to chamber 69, acting as a “dry” seal element preventing the backflow of gases.

When the face plate 26 is bonded to the flow control module 16 and collection module 12, at least a first fluid flow passageway is defined from the entry port 18 on the flow control module 16, through sub-compartments 42 and 48, down through the arm 52, through chamber 54 and arm 60, into chamber 64, and up out though opening 70 into suction port 20, as shown generally along flow arrows F2, F3, F4, and F5. A positive pressure relief valve element is also included into the form of a ball 72 inside sub-compartment 74 above an opening 76. Opening 76 is in communication with the suction flow and suction pressures present inside of chamber 69. The sub-compartment 74 has the opening 30 exposed to ambient air external to the flow control module 16. Thus when the suction pressure inside chamber 69 is sufficient, the ball element 72 rests down against the valve seat 76 closing the valve. But when pressure inside the flow control module 16 and chamber 69 (reflective of pressures existing throughout the apparatus 10 and also inside the patient) is too high and exceeds a pre-determined positive pressure threshold, the ball element 72 will separate from the valve seat 76 and excessive positive pressure inside flow control module 16 will exit through hole 30 to the ambient room air to reduce the pressure therein.

The pressure regulator module 24 is shown to be sealingly coupled or attached to the flow control module 16 as shown in FIG. 3, through the pressure regulation flow intake port 80. The pressure regulator module 24 has an ambient air intake port 82 though which room air at non-suction pressures can be sucked though the pressure regulator 24. The pressure regulator 24 includes a user-adjustable dial element 84 which can be accessed through a hole fitted in the face plate (not shown). The pressure regulator 24 can have any number of known valve assemblies therein, such as, for example, a valve seat and poppet element (not shown) that are biased by a spring element in compression. The spring element and valve are calibrated to open and close relative to certain differentials of pressure between the exterior of the device and in the interior of the flow control module 16 as transmitted through channel or compartment 90. When the pressure regulator 24 is open, room air is allowed into the flow control module 16 to equalize pressures and flows along a second flow pathway in said module along arrow F6 as shown. This ‘pressure regulation flow’ mixes with the suction flow F5 just before the suction port 20 to control the operating pressures inside the device 10, such as in the collection chamber 40.

FIG. 4 is a perspective view illustrating a flow control module of the present invention in accordance with one embodiment. An access hole 92 is provided in a front panel 94 of the flow control module 16, through which the adjustable controls of the pressure regulation module 24 (not shown) would be accessible when the pressure regulation module is assembled with the flow control module 16. A horizontal shelf 96 is also shown in arm 60 having an opening 98 at the back end of shelf 96 away from the front panel end of the flow control module, and provides the means for flow F4 to enter through to opening 62 and on into chamber 64. The shelf 96 and opening 98 it defines together make up a tortuous flow path in arm 60 that prevents liquids in chamber 54 from passing upwards into more downstream portions of the device. As used herein, the term “downstream” shall mean further along in the device's flow pathways, such as along suction flow pathways F2, F3, F4 or F5 shown in FIGS. 3 and 4. As used herein, “upstream” shall mean the opposite of downstream. Thus, check valve element made up of ball 46 and seat 44 is downstream of the entry port 18, as suction flow will tend to flow in the direction away from entry port 18 through chamber 48 and into arm 52.

To assemble the device, the collection module 12 is formed from a first base mold. The flow control module 16 is formed from a second base mold. The adjustable pressure regulation module 24 is separately made and assembled. The face plate 26 is separately made, and is provided with a variety of labels, markers and gradations, with corresponding transparent window elements, such as to read the water level in the arm 52, air bubbles in the air leak meter in chamber 54, the setting of pressure regulator dial 84, and the like. The collection module 12 and flow control module 16 are bonded or attached to the face plate 26. The adjustable pressure regulation module 24 is coupled or bonded to the flow control module 16 to allow regulation of the pressure inside the collection module 12 and flow control module 16. When the face plate 26 is bonded onto the flow control module 16 and collection chamber module 12, the various compartments 40, 42, 48, 52, 54, 60, 64, 69, and 90 are closed off to form closed chambers which define flow pathways or pressure holding spaces, thereby creating the flow and pressure control functionalities discussed herein.

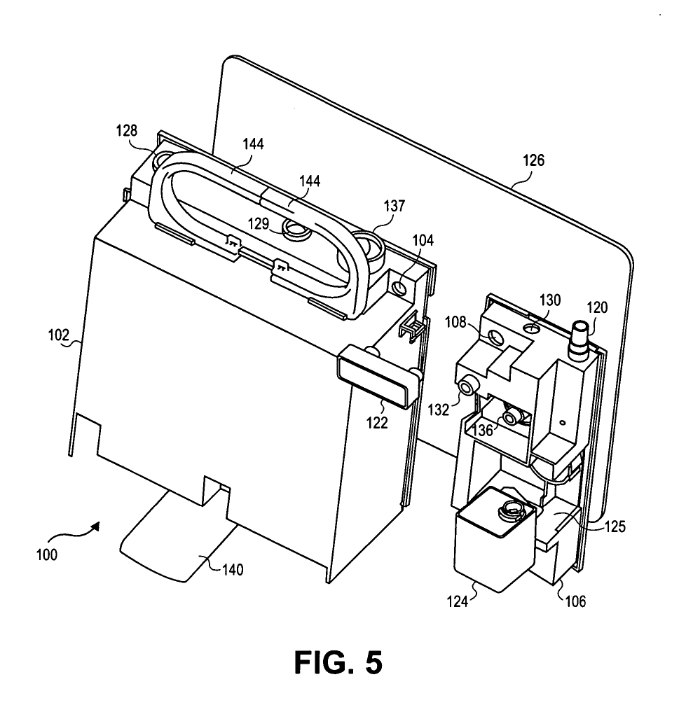

The size and scale of the various components for the modular assembly disclosed herein can be varied depending on the needs of the patient. Adult sizes having collection chambers with a volume of about 2000 cc are possible, while pediatric sizes having collection chambers with a volume of 500 cc are possible as well. FIG. 5 is a perspective view illustrating the separate components of a modular chest drainage device prior to final assembly, according to another embodiment of the invention. A modular chest drainage device 100 includes a collection module 102 defining a fluid collection chamber inside of it and having an exit port 104 for transmitting a suction flow out of the collection chamber. A ‘flow control’ module 106 defines an entry port 108 for receiving the suction flow from the collection chamber, a suction port 120 for coupling to a suction source (not shown), and a pressure regulation flow intake port (similar to that shown for the pressure regulator 24 shown in FIGS. 3 and 4). A flow coupling 122 is provided between the exit port 104 and the entry port 108. A pressure regulation module 124 is sealingly coupled to the pressure regulation flow intake port on the flow control module 106 and can be positioned in a c-shaped enclosure 125 defined by the walls and geometry of the flow control module 106 as shown in FIG. 5. A face plate 126 is provided, wherein the collection module 102 and flow control module 106 are brought side by side and are attached, bonded, or otherwise coupled to said face plate 126, which can have any number of openings therein for viewing or controlling functioning elements of the assembly 100, similar to those elements discussed above with respect to the assembly 10.

The collection module 102 includes a patient fluid intake port 128 for receiving fluids from a patient's body. A catheter, tube, or similar device can be coupled to the fluid intake port 128 in a variety of ways as is well known in the art. A second patient intake port 129 can also be provided in the form of a punch-out port, if multiple bodies need to be drained and coupled to the assembly for withdrawal of fluid. An ambient air port 130 is included on the flow control module 106 as part of a positive pressure relief valve element therein. A filling valve 132, such as a grommet or needle-less fill valve with a lure type fitting, is provided on the flow control module 106 for injecting fluids into the module for filling a manometer chamber or water seal chamber that is needed to control the backflow of gases and to indicate pressure, flow, or breathing, as further explained below. An automatic high negativity pressure relief valve 136 is also provided on the flow control module 106 to prevent excessive negative pressures from building in the device. In addition, a manually actuated high negative pressure relief valve 137 can be provided on the collection module 102. For ease of handling and stability, the collection module 102 can also include on its bottom surface an adjustable floor stand 140 and a pair of removable handle elements 144.

The great advantage of the modular assembly disclosed herein is that it reduces the size and complexity of the base molds needed to make the various modules. The molds, as well as any changes or repairs, will be less complex and expensive. And if one element in the overall apparatus is faulty or needs to be changed, that element can be more easily redesigned and manufactured, thus providing the true modularity of the chest drainage device of the present invention.

The many features and advantages of the invention are apparent from the detailed specification, and thus, it is intended by the appended claims to cover all such features and advantages of the invention which fall within the true spirit and scope of the invention. Further, since numerous modifications and variations will readily occur to those skilled in the art, it is not desired to limit the invention to the exact construction and operation illustrated and described, and accordingly, all suitable modifications and equivalents maybe resorted to, falling within the scope of the invention.

What is claimed is:

1. A modular chest drainage assembly, comprising:
a collection module formed from a first base mold, the collection module defining a collection chamber having a patient fluid intake port;
a flow control module formed from a second base mold;
a separate flow coupling attachable to the collection module and flow control module to place the two modules in fluid communication;
a pre-assembled pressure regulation module coupled to the flow control module to regulate the pressure inside the collection module and flow control module, and
a face plate, to which the collection module and flow control module are bonded such that the flow control module defines a first fluid flow pathway from the flow coupling to a suction port on the flow control module, for transmitting a suction flow from the collection chamber to the suction port.

2. The modular chest drainage assembly of claim 1, further comprising:
an air leak indicator and metering element disposed in the first fluid flow pathway.

3. The modular chest drainage assembly of claim 1, further comprising:
a water seal element disposed in the first fluid flow pathway to prevent gases from flowing from the flow control module to the collection module.

4. The modular chest drainage assembly of claim 1, further comprising:
a breathing indicator manometer column defined in the first fluid flow pathway below an entry port on the flow control module where the flow coupling is attached to said module, the manometer column being fillable with fluid injected through a needleless fill valve disposed on the flow control module.

5. The modular chest drainage assembly of claim 4, further comprising:
a check valve element disposed in the first fluid flow pathway downstream of the entry port and upstream of the breathing indicator manometer, having a ball element disposed to close a valve seat defined by the flow control module above the ball element when the pressure in the first fluid flow pathway exceeds a pre-determined relative threshold relative to the pressure in the collection chamber.

6. The modular chest drainage assembly of claim 1, further comprising:
a high negative pressure relief valve disposed in the flow control module, placing the first fluid flow pathway in fluid communication with ambient air outside the flow control module when the pressure inside the first fluid flow pathway exceeds a pre-determined limit of negative pressure.

7. The modular chest drainage assembly of claim 1, further comprising:
a positive pressure relief valve element disposed in the flow control module, having a ball element disposed above a valve seat in communication with the first fluid flow pathway, and having a ambient air port on an exterior surface of the flow control module, the ball element being separated from the valve seat to open the positive pressure relief valve element when the pressure inside the first fluid flow pathway exceeds a pre-determined limit of positive pressure.

8. The modular chest drainage assembly of claim 1, further comprising:
a body fluid re-infusion port defined by the collection module for transferring body fluids collected in the collection chamber from a patient from in the chest drainage assembly back to the patient.

9. The modular chest drainage assembly of claim 1,
wherein the flow control module defines an enclosure into which the pre-assembled pressure regulation module is disposed when coupled to the flow control module.

10. A modular chest drainage device, comprising:
a collection module defining a fluid collection chamber having an exit port for transmitting a suction flow out of the collection chamber;
a flow control module defining an entry port for receiving the suction flow from the collection chamber, a suction port for coupling to a suction source, and a pressure regulation flow intake port;
a flow coupling between the exit port and the entry port;
a pressure regulation module sealingly coupled to the pressure regulation flow intake port, having an adjustable valve assembly for regulating a pressure regulation flow into the flow control module from an ambient air intake port, and
a face plate, to which the collection module and flow control module are attached to form (i) a first fluid flow pathway from the entry port to the suction port for transmitting the suction flow from the collection chamber to the suction port, and (ii) a second fluid flow pathway from the pressure regulation flow intake port to the suction port for transmitting the pressure regulation flow, wherein the mixing of the suction flow and pressure regulation flow provides for regulation of pressure inside the collection chamber.

11. The modular chest drainage device of claim 10, further comprising:
an air leak indicator and metering element disposed in the first fluid flow pathway.

12. The modular chest drainage device of claim 10, further comprising:
a water seal element disposed in the first fluid flow pathway to prevent gases from flowing from the flow control module to the collection module.

13. The modular chest drainage device of claim 10, further comprising:
a breathing indicator manometer column defined in the first fluid flow pathway below the entry port, the manometer column being fillable with fluid injected through a needleless fill valve disposed on the flow control module.

14. The modular chest drainage device of claim 10, further comprising:
a check valve element disposed in the first fluid flow pathway downstream of the entry port and upstream of the breathing indicator manometer, having a ball element disposed to close a valve seat defined by the flow control module above the ball element when excessive pressure exists in the first fluid flow pathway.

15. The modular chest drainage device of claim 10, further comprising:
a high negative pressure relief valve disposed in the flow control module, placing the first fluid flow pathway in fluid communication with ambient air outside the flow control module when the pressure inside the first fluid flow pathway exceeds a pre-determined limit of negative pressure.

16. The modular chest drainage device of claim 10, further comprising:
a positive pressure relief valve element disposed in the flow control module, having a ball element disposed above a valve seat in communication with the first fluid flow pathway, and having a ambient air port, the ball element being separated from the valve seat to open the positive pressure relief valve element when the pressure inside the first fluid flow pathway exceeds a pre-determined limit of positive pressure.

17. The modular chest drainage device of claim 10, further comprising:
a re-infusion port defined by the collection module for transferring body fluids collected in the collection chamber from a patient from the chest drainage device back to the patient.

18. The modular chest drainage assembly of claim 10,
wherein the flow control module defines an enclosure into which the pressure regulation module is disposed when coupled to the flow control module.

19. A method of assembling a chest drainage device, comprising:
forming a collection module from a first base mold, the collection module defining a collection chamber having a patient fluid intake port;
forming a flow control module from a second base mold;
assembling an adjustable pressure regulation module;
forming a face plate;
bonding the collection module and flow control module to the face plate such that the flow control module defines a first fluid flow pathway from a flow entry port to a suction port on the flow control module, for transmitting a suction flow from the collection chamber to the suction port; and
coupling the adjustable pressure regulation module to the flow control module to allow regulation of the pressure inside the collection module and flow control module.

20. The method of assembling a chest drainage device of claim 19, further comprising:
forming a flow coupling;
attaching the flow coupling to the collection module and flow control module to place the two modules in fluid communication such that suction flow is transmitted from the collection chamber into the first fluid flow pathway in the flow control module.
